(19) United States
(12) Patent Application Publication
(10) Pub. No.: US 2019/0168158 A1
(43) Pub. Date: Jun. 6, 2019

(54) HYDROGEN PEROXIDE VAPOR DETOXIFYING SYSTEM

(71) Applicant: KOREA INSTITUTE OF SCIENCE AND TECHNOLOGY, Seoul (KR)

(72) Inventors: Kang Bong LEE, Seoul (KR); Yeong Cheon KIM, Seoul (KR); Yun Sik NAM, Seoul (KR); Myeong Gi KIM, Seoul (KR); Jin Oh JEON, Seoul (KR); Jae Eun KIM, Chungcheongnam-do (KR)

(21) Appl. No.: 15/910,007

(22) Filed: Mar. 2, 2018

(30) Foreign Application Priority Data
Dec. 4, 2017 (KR) 10-2017-0165125

(51) Int. Cl. B01D 53/86; B01J 23/42; B01J 35/04; A61L 2/20

(52) U.S. Cl. CPC B01D 53/869; B01D 53/8671; B01J 23/42; B01J 35/04; A61L 2202/13; B01D 2255/2073; B01D 2255/1021; B01D 2255/904; B01D 2257/93; A61L 2/208

(57) ABSTRACT

Disclosed is a hydrogen peroxide vapor detoxifying system, which includes a hydrogen peroxide vapor suction unit configured to suck a hydrogen peroxide vapor in a target space; a hydrogen peroxide vapor detoxifying unit configured to decompose the sucked hydrogen peroxide vapor; and a vapor discharging unit configured to discharge a decomposition product generated by decomposing the hydrogen peroxide vapor.

### CROSS-REFERENCE TO RELATED APPLICATION

This application claims priority to Korean Patent Application No. 10-2017-0165125, filed on Dec. 4, 2017, and all the benefits accruing therefrom under 35 U.S.C. § 119, the contents of which in its entirety are herein incorporated by reference.

### BACKGROUND

### 1. Field

The present disclosure relates to a hydrogen peroxide vapor detoxifying system, and more particularly, to a system for sucking, decomposing and detoxifying hydrogen peroxide vapor sprayed in a room or a sealed space for sterilization.

### 2. Description of the Related Art

Generally, when hydrogen peroxide vapor (HPV) is generated for sterilization, hydrogen peroxide molecules generate active oxygen (oxygen free radical) to serve as a disinfectant that decomposes and sterilizes various fungi, bacteria, viruses and spores.

However, since hydrogen peroxide vapor is a toxic compound harmful to human body, it is necessary to decompose hydrogen peroxide vapor remaining after use into water and oxygen by a decomposition catalyst, for the purpose of detoxification.

In other words, if the hydrogen peroxide vapor comes into contact with the human body or is inhaled by the human body, it may cause skin irritation or inflammation and fatal damages to respiratory organs such as nose, throat and lung, and there is a risk of corneal injury and blindness.

Thus, the hydrogen peroxide vapor should be rapidly decomposed for detoxification after being used in a confined space for sterilization.

### RELATED LITERATURES

### Patent Literature

Patent Literature 1: KR Patent Application publication No. 10-2016-0083423

Patent Literature 2: KR Patent Application publication No. 10-2015-0061203

### SUMMARY

Generally, it would be the simplest method to dilute hydrogen peroxide vapor with air, but this may contaminate the surrounding air with hydrogen peroxide, which may cause toxic damage due to secondary pollution. Thus, hydrogen peroxide vapor should be decomposed and removed in the room.

For example, as shown in FIG. 11, Korean Unexamined Patent Publication No. 10-2016-0083423 (Prior Art 1) discloses a system 1000 for sterilizing hydrogen peroxide vapor vaporized through a heating unit 1001 by using a discharging unit 1002, and the system disclosed in Prior Art 1 detoxifies hydrogen peroxide vapor, exposed to a sterilized air, by means of ventilation.

Also, as shown in FIG. 12, Korean Unexamined Patent Publication No. 10-2015-0061203 (Prior Art 2) does not consider a method of effectively detoxifying a discharged sterilizing liquid at all.

So far, for decomposing and detoxifying hydrogen peroxide vapor sprayed to a target space, required components are installed in the room together with a hydrogen peroxide vapor generation device to perform sterilization after sterilization. At this time, it takes much time to lower a hydrogen peroxide vapor concentration in the indoor space to a limit concentration, which deteriorates the efficiency of equipment utilization and causes problems in recycling a decomposition catalyst and deteriorates efficacy.

Thus, in order to efficiently decompose and detoxify hydrogen peroxide vapor, it is needed to develop a hydrogen peroxide vapor detoxifying system, which may be operated conveniently, detoxify very efficiently, enables recycling of a hydrogen peroxide vapor decomposing catalyst, and not cause a pressure drop phenomenon. In one aspect, there is provided a hydrogen peroxide vapor detoxifying system, comprising: a hydrogen peroxide vapor suction unit configured to suck a hydrogen peroxide vapor in a target space; a hydrogen peroxide vapor detoxifying unit configured to decompose the sucked hydrogen peroxide vapor; and a vapor discharging unit configured to discharge a decomposition product generated by decomposing the hydrogen peroxide vapor.

In addition, the vapor suction unit of the present disclosure may further include a control unit configured to adjust a sucked amount of the hydrogen peroxide vapor.

In addition, the vapor detoxifying unit of the present disclosure may include: a housing; a honeycomb structure accommodated in the housing; and a preheater accommodated in the housing and disposed before the honeycomb structure in a direction of sucking the hydrogen peroxide vapor.

In addition, the housing of the present disclosure may further include: a sensor configured to measure temperature of the preheater; and a temperature regulating unit configured to control the temperature of the preheater.

In addition, the preheater of the present disclosure may include a plurality of preheating plates disposed to be spaced apart from each other, and the preheater may preheat the sucked hydrogen peroxide vapor to a temperature range of 50° C. to 150° C.

In addition, the honeycomb structure of the present disclosure may be provided in plural, and the plurality of honeycomb structures may be arranged in series. Also, the honeycomb structure may have a cylindrical shape and include a structural part disposed in the cylindrical honeycomb structure in a mesh form.

In addition, the structural part of the present disclosure may be coated with at least one catalyst selected from platinum, iron oxide and nickel oxide.

In addition, a ring may be disposed at an outer circumference of the cylindrical honeycomb structure to fix the honeycomb structure in the housing.

In addition, the hydrogen peroxide vapor discharging unit of the present disclosure may include a filtering unit configured to filter the decomposition product generated by decomposing the hydrogen peroxide vapor.

The present disclosure may provide a hydrogen peroxide vapor detoxifying system that solves various issues such as air flow, pressure drop, catalyst recycling, size and weight of equipment including an air preheater, and portability.

Also, the present disclosure may increase the hydrogen peroxide vapor decomposing and detoxifying efficiency and simplify the equipment by using a catalyst formed with a honeycomb structure.

In addition, the present disclosure may provide a convenient hydrogen peroxide vapor detoxifying system that may quickly cope with situations.

### BRIEF DESCRIPTION OF THE DRAWINGS

FIG. 1 is a schematic perspective view showing an interior of a hydrogen peroxide vapor detoxifying system according to an embodiment of the present disclosure, observed in a direction of sucking a hydrogen peroxide vapor.

FIG. 2 shows the hydrogen peroxide vapor detoxifying system of FIG. 1, observed in a direction of discharging a decomposition product generated by decomposing the hydrogen peroxide vapor.

FIG. 3 is a schematic perspective view showing an interior of a hydrogen peroxide vapor detoxifying unit according to an embodiment of the present disclosure, where a single honeycomb structure is disposed.

FIG. 4 is a schematic perspective view showing an interior of a hydrogen peroxide vapor detoxifying unit according to an embodiment of the present disclosure, where two honeycomb structures are arranged in series.

FIG. 5 is a schematic perspective view showing an exterior of a hydrogen peroxide vapor detoxifying unit according to an embodiment of the present disclosure, where a preheater and a sensor for measuring temperature of the preheater are provided.

FIG. 6 is a schematic perspective view showing an interior of the hydrogen peroxide vapor detoxifying unit of FIG. 5, where a preheater is disposed before the honeycomb structure in a direction of sucking the hydrogen peroxide vapor.

FIG. 7 shows a comparative example where a manganese dioxide catalyst is filled instead of the honeycomb structure in the hydrogen peroxide vapor detoxifying unit.

FIG. 8 is a graph showing a hydrogen peroxide vapor decomposition efficiency of the hydrogen peroxide vapor detoxifying system according to the embodiment of the present disclosure and the detoxifying system according to the comparative example of FIG. 7, respectively.

FIG. 9 is a graph showing a decompression amount of air flow in the hydrogen peroxide vapor detoxifying system according to the embodiment of the present disclosure and the detoxifying system according to the comparative example of FIG. 7, respectively.

FIG. 10 is a graph showing weight and volume of the hydrogen peroxide vapor detoxifying system according to the embodiment of the present disclosure and the detoxifying system according to the comparative example of FIG. 7, respectively.

FIG. 11 schematically shows a conventional system for injecting hydrogen peroxide vapor into a sterilization target space.

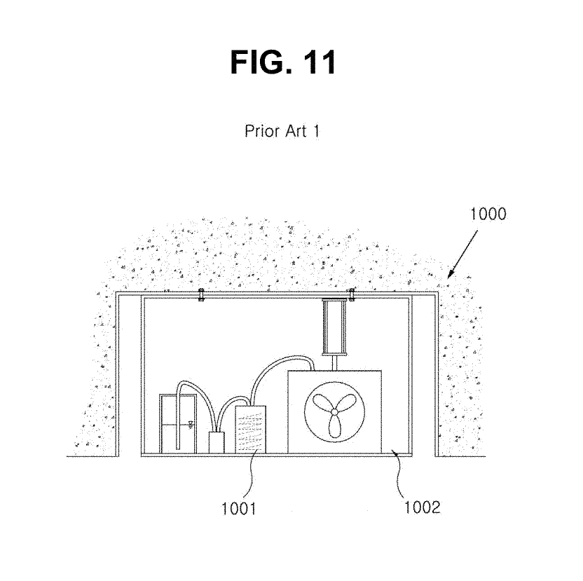

FIG. 12 is a block diagram for illustrating a conventional hydrogen peroxide catalyst decomposing method.

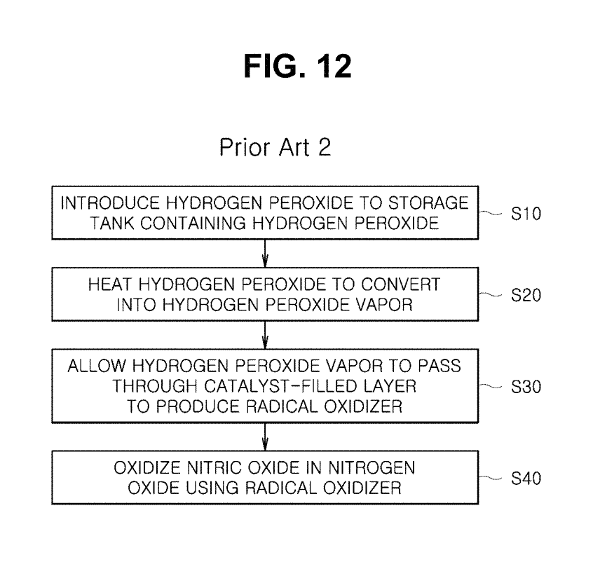

### DETAILED DESCRIPTION

Hereinafter, a hydrogen peroxide vapor detoxifying system 100 according to an embodiment of the present disclosure will be described with reference to the accompanying drawings.

Prior to explanation, it should be understood that like elements are denoted by like reference signs in various embodiments and representatively described in a single embodiment, and different elements are described in other embodiments.

Meanwhile, in order to clearly explain the operation of the hydrogen peroxide vapor detoxifying system 100 according to an embodiment of the present disclosure, the flow of the hydrogen peroxide vapor is indicated by an arrow.

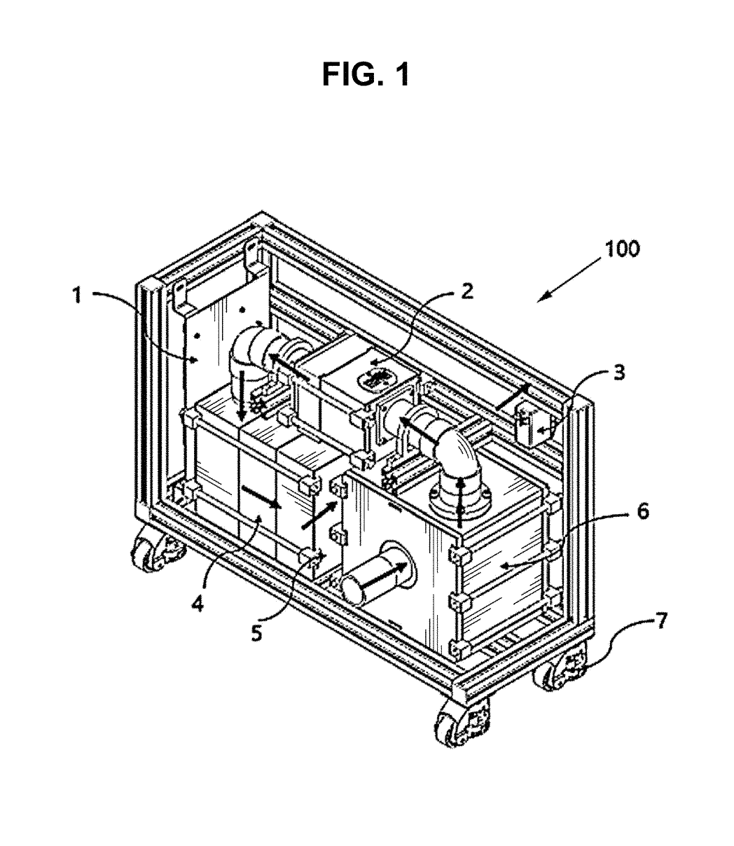

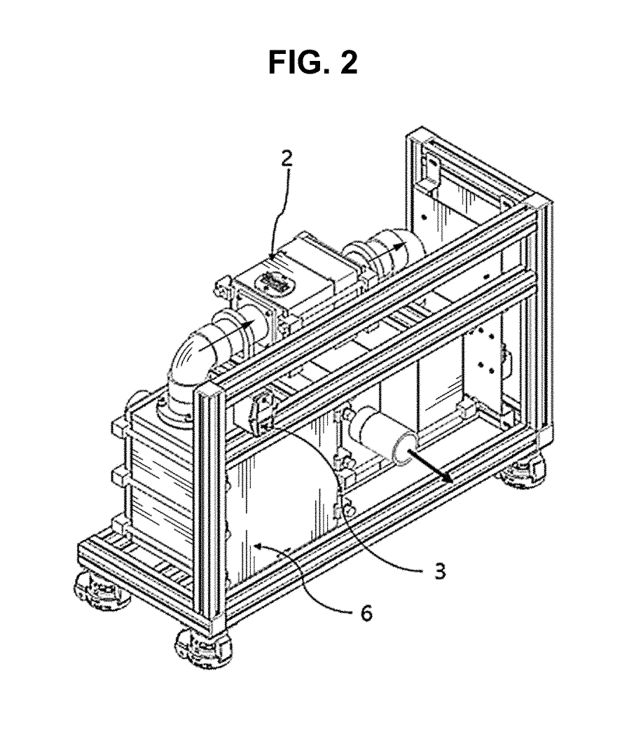

FIGS. 1 and 2 are perspective views schematically showing an interior of a hydrogen peroxide vapor detoxifying system 100 according to an embodiment of the present disclosure. Here, FIG. 1 is a view observed in the direction of sucking the hydrogen peroxide vapor, and FIG. 2 is a view observed in the direction of discharging a decomposition product generated by decomposing the hydrogen peroxide vapor.

As shown in FIGS. 1 and 2, the hydrogen peroxide vapor detoxifying system 100 according to an embodiment of the present disclosure includes a vapor suction unit 6 for sucking a hydrogen peroxide vapor discharged into a target space, a hydrogen peroxide vapor detoxifying unit 2 for decomposing the sucked hydrogen peroxide vapor, and a vapor discharging unit 4 for discharging a decomposition product generated by decomposing the hydrogen peroxide vapor. These components are connected to a delivery pipe through which the hydrogen peroxide vapor and the decomposition product of the hydrogen peroxide vapor flow.

Specifically, the hydrogen peroxide vapor suction unit 6 may control a sucked amount of the hydrogen peroxide vapor by means of a control unit 1.

In addition, the hydrogen peroxide vapor detoxifying system 100 may further include a power supply 3 of the hydrogen peroxide vapor detoxifying system 100 and a carrier 7 such as a wheel for convenient movement.

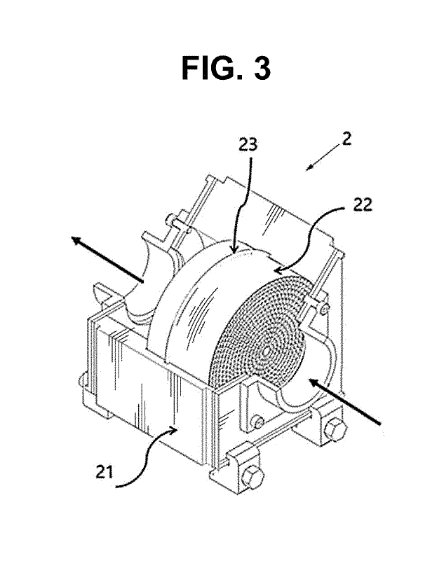

FIG. 3 is a perspective view schematically showing the interior of the hydrogen peroxide vapor detoxifying unit 2 according to an embodiment of the present disclosure, and FIG. 3 shows that a single honeycomb structure 22 is disposed therein.

As shown in FIG. 3, the hydrogen peroxide vapor detoxifying unit 2 includes a housing 21 and a honeycomb structure 22 accommodated in the housing 21.

In the present disclosure, the hydrogen peroxide vapor detoxifying unit 2 may have various configurations depending on the number of catalyst units, the connection state, the type of catalyst, and the presence or absence of an air preheater in a target space.

For example, as shown in FIG. 3, in the vapor detoxifying unit 2, the sucked hydrogen peroxide vapor passes through the single honeycomb structure 22 and is discharged to the vapor discharging unit 4.

Meanwhile, the hydrogen peroxide vapor discharging unit 4 may include a filtering unit for filtering the decomposition product gas generated by decomposing the hydrogen peroxide vapor.

In addition, the honeycomb structure 22 may have a cylindrical shape, and a catalyst for decomposing the hydrogen peroxide vapor is coated on a structural part disposed at the inside of the cylindrical honeycomb structure 22 in a mesh form. The catalyst may employ a platinum (Pt) catalyst, and at least one catalyst selected from iron oxide or nickel oxide may be coated on the structural part to enhance the hydrogen peroxide vapor removal efficiency.

Meanwhile, a ring 23 for fixing the position of the honeycomb structure 22 inside the housing 21 may be disposed at an outer circumference of the cylindrical honeycomb structure 22, and a sealing silicone for preventing leakage of the hydrogen peroxide vapor may be applied at each edge of the housing 21.

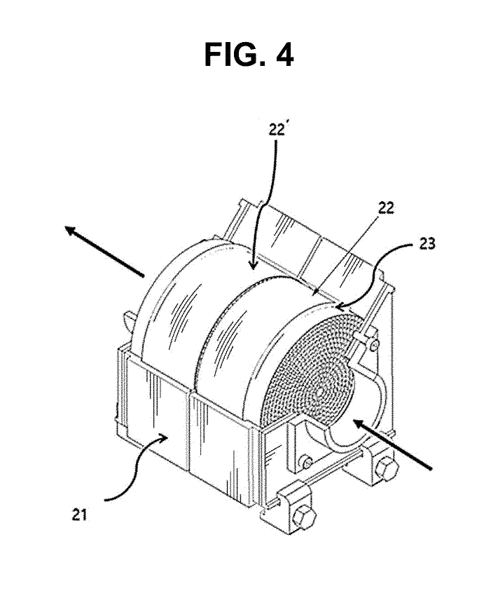

FIG. 4 is a perspective view schematically showing the interior of a hydrogen peroxide vapor detoxifying unit 2 according to another embodiment of the present disclosure, in which two honeycomb structures 22 and 22′ are arranged in series.

As shown in FIG. 4, since two honeycomb structures 22 and 22′ are provided for decomposing hydrogen peroxide, the surface area where the hydrogen peroxide vapor comes into contact with the catalyst is larger than that of the vapor detoxifying unit 2 having a single honeycomb structure. Thus, it is possible to increase the hydrogen peroxide vapor decomposition efficiency as much.

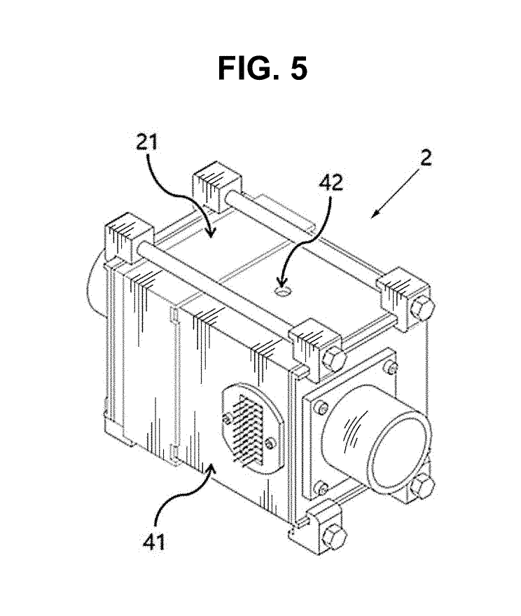

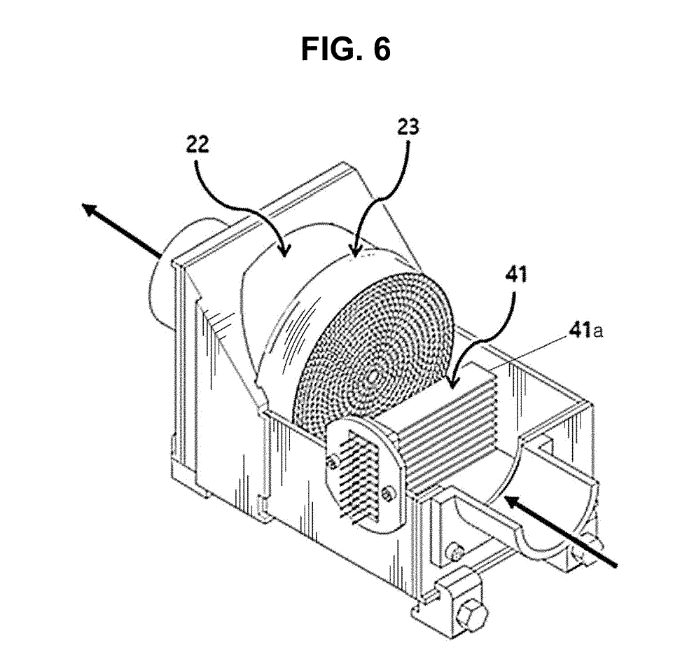

FIG. 5 is a perspective view schematically showing the exterior of the hydrogen peroxide vapor detoxifying unit 2 according to an embodiment of the present disclosure. Here, FIG. 5 shows a preheater 41 and a sensor 42 for measuring the temperature of the preheater, and FIG. 6 shows that the preheater 41 is disposed before the honeycomb structure 22 in the direction of sucking the hydrogen peroxide vapor in the hydrogen peroxide vapor detoxifying unit 2 of FIG. 5.

As shown in FIGS. 5 and 6, the hydrogen peroxide vapor introduced through the preheater 41 may be preheated in advance in order to increase the hydrogen peroxide vapor decomposition efficiency, and the temperature of the preheater 41 may be measured by using the temperature sensor 42 located at the top end of the housing 21 so that the temperature regulating unit regulates the temperature of the preheater 41 by controlling the control unit 1 described above. The preheater 41 may be preheated to a temperature range of 50° C. to 150° C. in consideration of the activating temperature of the hydrogen peroxide vapor.

In addition, the preheater 41 may include a plurality of preheating plates 41a spaced apart from each other to maximize the contact area between the preheating plates 41a and the hydrogen peroxide vapor before the hydrogen peroxide vapor enters the honeycomb structure 41, thereby improving the preheating efficiency.

Generally, it takes much time to decompose hydrogen peroxide vapor. Here, by using the preheater 41 described above, it is possible to enhance the hydrogen peroxide vapor decomposition efficiency as much as the activating temperature of the hydrogen peroxide vapor with the catalyst of the honeycomb structure 22 increases.

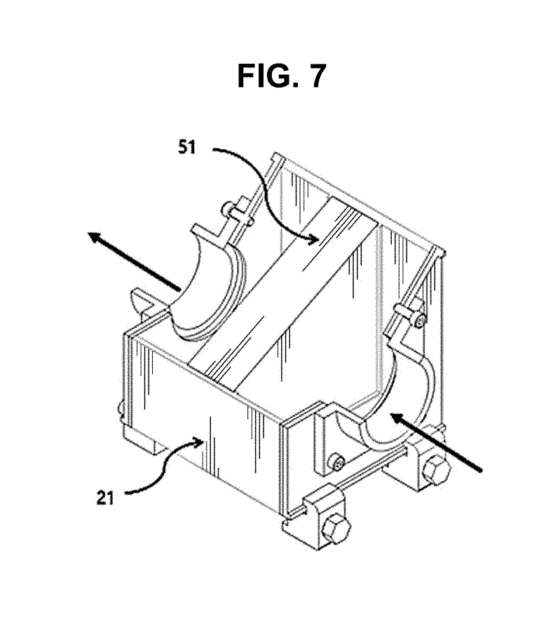

Hereinafter, an experiment for testing the hydrogen peroxide vapor decomposition efficiency using the hydrogen peroxide vapor detoxifying system 100 according to an embodiment of the present disclosure will be explained. As a comparative example of the experiment, as shown in FIG. 7, a detoxifying system filled with a manganese dioxide catalyst 51, instead of the honeycomb structure 22 according to an embodiment of the present disclosure is used.

### Experiment 1: Measurement of the Hydrogen Peroxide Vapor Decomposition Efficiency

The detoxifying efficiency of each hydrogen peroxide vapor is measured according to the type of the vapor detoxifying unit 2.

Experiments are conducted using an example where a single honeycomb structure 22 is disposed, an example where two honeycomb structures 22 are arranged in series, an example where the preheater 41 is provided together, and a comparative example where a manganese dioxide catalyst is used.

The experiments are carried out at room temperature (25° C.), and the hydrogen peroxide vapor concentration at which the hydrogen peroxide vapor concentration is introduced into the detoxifying system 100 is fixed at 1000 ppm.

In addition, a concentration measuring device is respectively installed at an inlet and an outlet to measure the hydrogen peroxide concentration. The concentration measuring device is PortaSens II manufactured by ATI, and the manganese dioxide catalyst 51 used in the comparative example has a weight of 400 g.

In addition, a pump for sucking air in the target space has a sucking capacity of 50 m³/hr, and in the example where the preheater 41 of the present disclosure is used, the temperature of the preheater 41 is kept at 70° C.

In addition, for the structural stability and accurate performance measurement of the hydrogen peroxide vapor detoxifying system, the performance of the system is measured 15 minutes after starting the system. A power source used is a voltage of 300 W to 500 W per system.

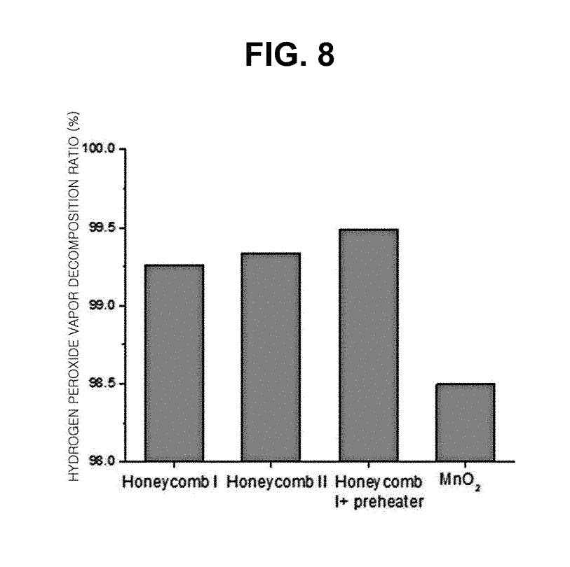

As shown in FIG. 8, the hydrogen peroxide vapor decomposition efficiency of the detoxifying system is very high, namely 99.26% and 99.36%, respectively, in the example where a single honeycomb structure employing a platinum catalyst is used and the example where two honeycomb structures are connected in series.

In addition, in the example where the preheater 41 is further disposed, it is found that the decomposition efficiency is 99.49%, which is improved as compared with the example where the preheater 41 is not used. This means that the concentration of 1000 ppm of the introduced hydrogen peroxide is lowered to about 5 ppm after the hydrogen peroxide passed through the system.

Meanwhile, the decomposition efficiency of the comparative example using manganese dioxide catalyst (MnO₂) is 99.40%. From this, it can be understood that the hydrogen peroxide vapor decomposition efficiency is very excellent when the honeycomb structure 22 and the preheater 41 are used together.

### Experiment 2: Measurement and Comparison of Decompression Amounts of Catalysts

The vapor transmission efficiency is measured using each system of the examples and the comparative examples.

As a specific measurement method, an atmospheric pressure before hydrogen peroxide vapor passes through each detoxifying unit and an atmospheric pressure after the hydrogen peroxide vapor passes through each detoxifying unit are measured and compared.

The experiments are carried out at room temperature (25° C.), and a flow rate of the supplied vapor is constant at 25 cm³/hr. A water column height (several millimeters (mmH₂O)) before and after passing through each detoxifying unit is checked and converted into the unit of pascal (Pa) that is a basic unit of pressure.

In the comparative example, 200 g of manganese dioxide catalyst is used, and the measurement is performed 15 minutes after the measurement device starts, in order to ensure the stability and accuracy of the experimental data.

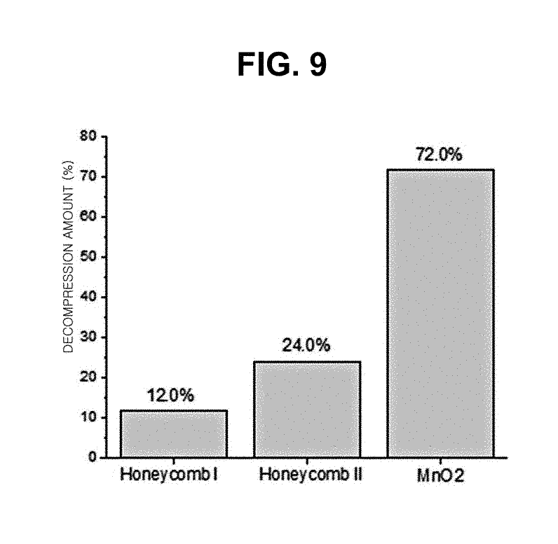

As shown in FIG. 9, the decompression amount is 12.00% and 24.00%, respectively, which are very low, when a single honeycomb structure is used (Honeycomb I) and when two honeycomb structures are used (Honeycomb II). In particular, when a single honeycomb structure is used, the vapor transmission ratio is very high.

In contrast, in the comparative example using manganese dioxide catalyst (MnO₂), the decompression amount is 72.00%, which means a very high pressure loss. This value is a very high value of about 6 times as compared with the examples of the present disclosure described above, and it is found that a pressure drop may occur as much.

### Experiment 3: Comparison of Physical Characteristics of Catalysts

Physical characteristics (weight and volume) of each detoxifying unit according to the examples of the present disclosure and the detoxifying unit according to the comparative example are measured. In order to measure a weight, a CAS electronic balance CUX6200HX is used, and in order to measure a volume, a method of measuring an increased volume immersed in a liquid is used.

The temperature of the liquid is measured at room temperature (25° C.). In addition, a measurement value obtained 2 minutes after the measurement is initiated is used in order to ensure the stability and accuracy of the experimental data.

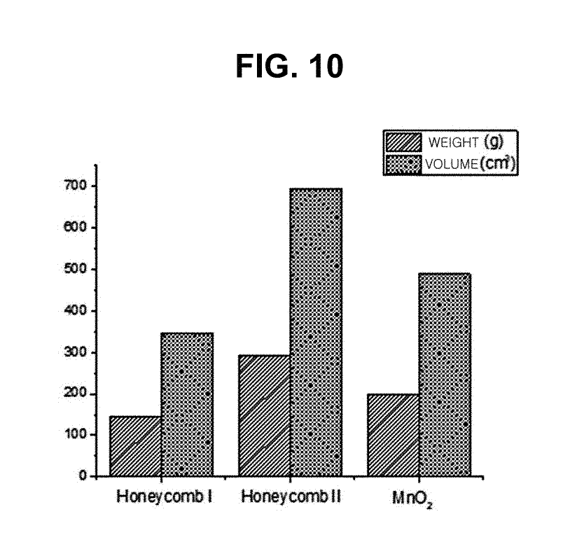

As shown in FIG. 10, when a single honeycomb structure is used (Honeycomb I), the weight and volume are 146 g and 347 cm³, respectively, which are lowest values. Thus, it can be found that this allows a very compact design due to small weight and volume.

In addition, when two honeycomb structures are arranged in series (Honeycomb II), the weight and volume are 292 g and 694 cm³, respectively, which are nearly twice as high as the above example (Honeycomb I).

In addition, in the comparative example using a manganese dioxide catalyst (MnO₂), it is found that the weight and volume are 200 g and 488 cm³, respectively, which are intermediate values of the examples of the present disclosure. Thus, by using the physical characteristics, it is possible to use an appropriate catalyst according to the characteristics of the space to be detoxified.

It will be understood by those skilled in the art that the present disclosure can be implemented in various ways without departing from the scope or essential characteristics of the present disclosure.

Therefore, it should be understood that the above embodiments are illustrative in all aspects and are not intended to limit the present disclosure to the embodiments. The scope of the present disclosure is defined by the appended claims, rather than the detailed description, and all changes or modifications derived from the claims and their equivalents should be construed as being included in the scope of the present disclosure.

The present disclosure provides a portable peroxide vapor detoxifying system that may overcome problems such as air flow, pressure drop and catalyst recycling, which may occur when a manganese dioxide catalyst is used, solve size and weight problems by designing the detoxifying unit very compactly, and be also portable.

In addition, the present disclosure provides a portable peroxide vapor detoxifying system that may completely detoxify a hydrogen peroxide vapor and utilize the hydrogen peroxide vapor in a target space again after the hydrogen peroxide vapor is used for sterilization, and that is capable of completely removing the hydrogen peroxide vapor so that the human body is not affected by a residual hydrogen peroxide vapor.

What is claimed is:

1. A hydrogen peroxide vapor detoxifying system, comprising:
a hydrogen peroxide vapor suction unit configured to suck a hydrogen peroxide vapor in a target space;
a hydrogen peroxide vapor detoxifying unit configured to decompose the sucked hydrogen peroxide vapor; and
a vapor discharging unit configured to discharge a decomposition product generated by decomposing the hydrogen peroxide vapor.

2. The hydrogen peroxide vapor detoxifying system according to claim 1,
wherein the vapor suction unit further includes a control unit configured to adjust a sucked amount of the hydrogen peroxide vapor.

3. The hydrogen peroxide vapor detoxifying system according to claim 1, wherein the vapor detoxifying unit includes:
a housing;
a honeycomb structure accommodated in the housing; and
a preheater accommodated in the housing and disposed before the honeycomb structure in a direction of sucking the hydrogen peroxide vapor.

4. The hydrogen peroxide vapor detoxifying system according to claim 3, wherein the housing further includes:
a sensor configured to measure temperature of the preheater; and
a temperature regulating unit configured to control the temperature of the preheater.

5. The hydrogen peroxide vapor detoxifying system according to claim 3,
wherein the preheater includes a plurality of preheating plates disposed to be spaced apart from each other.

6. The hydrogen peroxide vapor detoxifying system according to claim 4,
wherein the preheater preheats the sucked hydrogen peroxide vapor to a temperature range of 50° C. to 150° C.

7. The hydrogen peroxide vapor detoxifying system according to claim 3,
wherein the honeycomb structure is provided in plural, and the plurality of honeycomb structures are arranged in series.

8. The hydrogen peroxide vapor detoxifying system according to claim 7,
wherein the honeycomb structure has a cylindrical shape and includes a structural part disposed in the cylindrical honeycomb structure in a mesh form.

9. The hydrogen peroxide vapor detoxifying system according to claim 8,
wherein the structural part is coated with at least one catalyst selected from platinum, iron oxide and nickel oxide.

10. The hydrogen peroxide vapor detoxifying system according to claim 8,
wherein a ring is disposed at an outer circumference of the cylindrical honeycomb structure to fix the honeycomb structure in the housing.

11. The hydrogen peroxide vapor detoxifying system according to claim 1,
wherein the hydrogen peroxide vapor discharging unit includes a filtering unit configured to filter the decomposition product generated by decomposing the hydrogen peroxide vapor.
